(19) United States
(12) Patent Application Publication
(10) Pub. No.: US 2002/0182231 A1
(43) Pub. Date: Dec. 5, 2002

(54) Methods of stem cell manipulation for immunotherapy

(76) Inventors: Mary Pat Moyer, San Antonio, TX (US); Robyn Alyse McMasters, San Antonio, TX (US); Gina Lorraine Cano, St. Louis, MO (US); Kandy Kay Hudson, San Antonio, TX (US)

Correspondence Address: Mary Pat Moyer, Suite B-200, 12000 Network Blvd., San Antonio, TX, 78249, US

(21) Appl. No.: 10/102,662

(22) Filed: Mar. 22, 2002

Related U.S. Application Data
(60) Provisional application No. 60/278,162, filed on Mar. 23, 2001.

(51) Int. Cl. A61K 39/00; C12Q 1/68; G01N 33/574

(52) U.S. Cl. 424/277.1; 435/7.23; 435/6

(57) ABSTRACT

Methods for the expansion of blood stem cells and their activation as dendritic cells in a scheme for adjuvant immune therapy of patients with cancer or other diseases. The utility of combining a trade secret formulation of INCELL's M3™ medium in the expansion and activation of immune system stem cells and differentiated cells, including dendritic cells, with subsequent immunoreactivity against tumor cell antigens is demonstrated. The patent also describes long-term culture and cryopreservation of cells that maintain their reactivity and functionality, suggesting their potential use as universal donor cells. Furthermore, it describes how a dendritic cell activation kit can be developed using the approaches and the cell and media tools described.

### II. CROSS-REFERENCE TO RELATED APPLICATIONS

This is a utility patent application that follows on provisional patent #60/278,162.

### III. STATEMENT REGARDING FEDERALLY SPONSORED RESEARCH OR DEVELOPMENT

Not applicable

### IV. REFERENCE TO A SEQUENCE LISTING, A TABLE, OR A COMPUTER PROGRAM LISTING COMPACT DISC APPENDIX

Not applicable

### V. BACKGROUND OF THE INVENTION

This discovery relates to human stem cells, their culture, and their potential clinical use for cancer immune therapy. Background art can be found in the cited literature citations and in the listed patents. Interest in the use of dendritic and other cells for immunotherapy has increased in recent years (e.g., 9). However, there is a need to develop more cost effective and readily used methods for applying this technology in the clinic. To that end, the methods described herein, which maximize the potential utility of harvested and banked progenitor and stem cells, and minimize the time, costs and handling during ex-vivo manipulation, solve many problems currently associated with current methods.

Allogeneic stem cell transplants and the infusion of donor lymphocytes have demonstrated a graft versus tumor effect (e.g., 5, 6, 8, 9). Although significant morbidity and mortality from graft versus host disease and infectious complications have offset the potential benefits of these approaches, in vitro expansion of cells decreases some of the problems with antigen expression, and validation of their bioactivity is also inherent in any improvement for use of these cells for therapeutic applications. Many strategies are under investigation to induce a graft versus tumor effect following autologous or allogeneic stem cell transplants. These have focused on augmentation of antigen non-specific defenses. Antitumor responses have been generated in various tumor models by: 1) in-vitro activation of lymphoid cells with cytokines, antibodies (CD3), or lectins; 2) direct in-vivo administration of cytokines to stimulate anti-tumor effector cells in vivo, or 3) a combination of these two approaches (e.g., 1, 3, 4, 14). It is anticipated that these approaches may also allow for the augmentation and maintenance of immune activation after cell therapies such as activated T-lymphocytes and dendritic cells.

It is possible that incubation of dendritic cells or other immune cells with the allogeneic or autologous tumor cells could induce a more potent immune response. For example, the use of necrotic cell death for antigen presentation during dendritic cell culture may expose danger signals to these antigen-presenting cells and increase the potential for immune activation. Proof of principle of this concept has been demonstrated in studies where dendritic cells exposed to necrotic tumor cells have shown potent T-cell responses and anti-tumor effects (10, 13). These studies have also demonstrated that dendritic cell maturation and activation is heightened by exposure to necrotic tumor cells when compared to apoptotic cells. Collectively, these methods and cellular tools are expected to provide ways to develop tumor-specific adjuvant immunotherapies.

### Literature Citations

1. Bachier, C., Teale J, Lanzkron S, Hougham M, Nanez A, Huerta J, Childs C, LeMaistre CF. 1998. Concurrent and Seuential Administration of Interleukin-2 (IL-2) and Granulocyte-Macrophage Colony Stimulating Factor and Autologous Stem Cell transplant (ASCT). Blood. 92:4550.

2. Bender, A., M. Sapp, G. Schuler, R. M. Steinman, and N. Bhardwaj 1996. Improved methods for the generation of dendritic cells from nonproliferating progenitors in human blood J Immunol Methods. 196:121-35.

3. Benyuenes, M., Massumoto C, York A, et al. 1993. Interleukin-2 with or without lymphokine activated killer cells as consolidative immunotherapy after autologous bone marrow transplantation for acute myelogenous leukemia. Bone Marrow Transplant. 12:159-163.

4. Fefer, A., M. C. Benyunes, C. Massumoto, C. Higuchi, A. York, C. D. Buckner, and J. A. Thompson 1993. Interleukin-2 therapy after autologous bone marrow transplantation for hematologic malignancies Semin Oncol. 20:41-5.

5 Gahrton, G., S. Tura, P. Ljungman, B. Belanger, L. Brandt, M. Cavo, B. Chapuis, A. De Laurenzi, T. de Witte, T. Facon, and et al. 1991. Allogeneic bone marrow transplantation in multiple myeloma using HLA-compatible sibling donors—an EBMT Registry Study Bone Marrow Transplant. 7:32.

6. Gahrton, G., S. Tura, P. Ljungman, J. Blade, L. Brandt, M. Cavo, T. Facon, A. Gratwohl, A. Hagenbeek, P. Jacobs, and et al. 1995. Prognostic factors in allogeneic bone marrow transplantation for multiple myeloma [see comments] J Clin Oncol. 13:1312-22.

7. Hart, D. N. 1997. Dendritic cells: unique leukocyte populations which control the primary immune response Blood. 90:3245-87.

8. Lockhorst, H., Schattenverg J J, Cornelissen J J, thomas L L M, Verdonck L F. 1997. Donor lympocyte infusions are effective in relapsed multiple myeloma after allogenic bone marrow transplantation. Blood. 90:4206.

9. McCann, J. 1997. Immunotherapy using dendritic cells picks up steam [news] J Natl Cancer Inst. 89:541-2.

10. Melcher, A., S. Todryk, N. Hardwick, M. Ford, M. Jacobson, and R. G. Vile 1998. Tumor immunogenicity is determined by the mechanism of cell death via induction of heat shock protein expression Nat Med. 4:581-7.

11. Morse, M. A., L. J. Zhou, T. F. Tedder, H. K. Lyerly, and C. Smith 1997. Generation of dendritic cells in vitro from peripheral blood mononuclear cells with granulocyte-macrophage-colony-stimulating factor, interleukin-4, and tumor necrosis factor-alpha for use in cancer immunotherapy [see comments] Ann Surg. 226:6-16.

12. Romani, N., D. Reider, M. Heuer, S. Ebner, E. Kampgen, B. Eibl, D. Niederwieser, and G. Schuler 1996. Generation of mature dendritic cells from human blood. An improved method with special regard to clinical applicability J Immunol Methods. 196:137-51.

13. Sauter, B., M. L. Albert, L. Francisco, M. Larsson, S. Somersan, and N. Bhardwaj 2000. Consequences of cell death: exposure to necrotic tumor cells, but not primary tissue cells or apoptotic cells, induces the maturation of immunostimulatory dendritic cells [see comments] J Exp Med. 191:423-34.

14. Simpson, C., C. A. Seipp, and S. A. Rosenberg 1988. The current status and future applications of interleukin 2- and adoptive immunotherapy in cancer treatment Semin Oncol Nurs. 4:132-41.

15. Tarte, K., Z. Y. Lu, G. Fiol, E. Legouffe, J. F. Rossi, and B. Klein 1997. Generation of virtually pure and potentially proliferating dendritic cells from non-CD34 apheresis cells from patients with multiple myeloma Blood. 90:3482-95.

Patents in the Field Some patents in the field of use are listed from the international patent search (PCT; www.wipo.org) or US Patent Search (www.uspto.gov) when “dendritic cells” and stem cells were used as key words to define records.

| Patent Number | Title |
|---|---|
| PCT Patents | |
| WO 01/88105 | Production of Dendritic Cells From Bone-Marrow Stem |
| | Cells Approach, sources of cells and methods differ |
| WO 01/34645 | Modulating IL-13 Activity Using Mutated IL-13 |
| | Molecules that are Antagonists or Agonists of IL-13 |
| WO 00/28000 | Method for Producing Dendritic Cells |
| WO 99/63050 | Method for Preparation and in Vivo Administration |
| | of Antigen Presenting Cell Composition |
| WO 99/31227 | Novel Peptide, apoEpl.B, Compositions and Uses |
| | Thereof |
| WO 97/32992 | Immortalized Hematopoietic Cell Lines, Cell System |
| | Thereof with Stromal Cells, in Vitro, Ex Vivo and in |
| | Vivo uses, and in Vitro Generation of Dendritic Cells |
| | and Macrophages |
| WO 97/12633 | Dendritic Cell Stimulatory Factor |
| WO 97/03703 | Adeno-Associated Viral Liposomes and Their Use in |
| | Transfecting Dendritic Cells to Stimulate Specific |
| | Immunity |
| US Pat. Nos. | |
| 6,340,461 | Superantigen Based Methods and Compositions for |
| | Treatment of Diseases |
| 6,184,436 | Transgenic Mice Expressing HIV-1 in Immune Cells |
| 6,130,316 | Fusion Proteins of Novel CTLA4/CD28 Ligands and |
| | Uses Thereof |
| 6,100,443 | Universal Donor Cells |
| 6,084,067 | CTLA4/CD28 Ligands and Uses Thereof |
| 6,010,853 | Siva Genes, Novel Genes Involved in CD2-Mediated |
| | Apoptosis |
| 5,705,732 | Universal Donor Cells |

### VI. BRIEF SUMMARY OF THE INVENTION

This discovery optimizes methods for the expansion of blood stem cells and the use of activated dendritic and other immune cells as adjuvant immunotherapy for cancer patients. This patent demonstrates the utility of combining a trade secret formulation of INCELL's M3™ medium in the expansion and activation of immune system stem cells and differentiated cells, including dendritic cells, with subsequent immunoreactivity against tumor cells. It also describes long-term culture and cryopreservation of cells that maintain their reactivity and functionality, suggesting their potential use as universal donor cells. Furthermore, it describes how a DC activation kit could be developed using the approaches and the cell and media tools described.

### VII. BRIEF DESCRIPTION OF THE SEVERAL VIEWS OF THE DRAWINGS

Not applicable

### VIII. DETAILED DESCRIPTION OF THE INVENTION

Modes of carrying out invention are described in the 2 studies detailed below and the example of a type of kit that might be developed. The studies include: Study #1: stem cell isolation and methods for activation and evaluation of dendritic cells; and Study #2: validation of a dendritic cell activation and function. This study includes validation of marker expression and DC activation after long-term culture and long-term cryopreservation.

Study #1: Isolation of Stem Cells and Activation of Dendritic Cells (DCs)

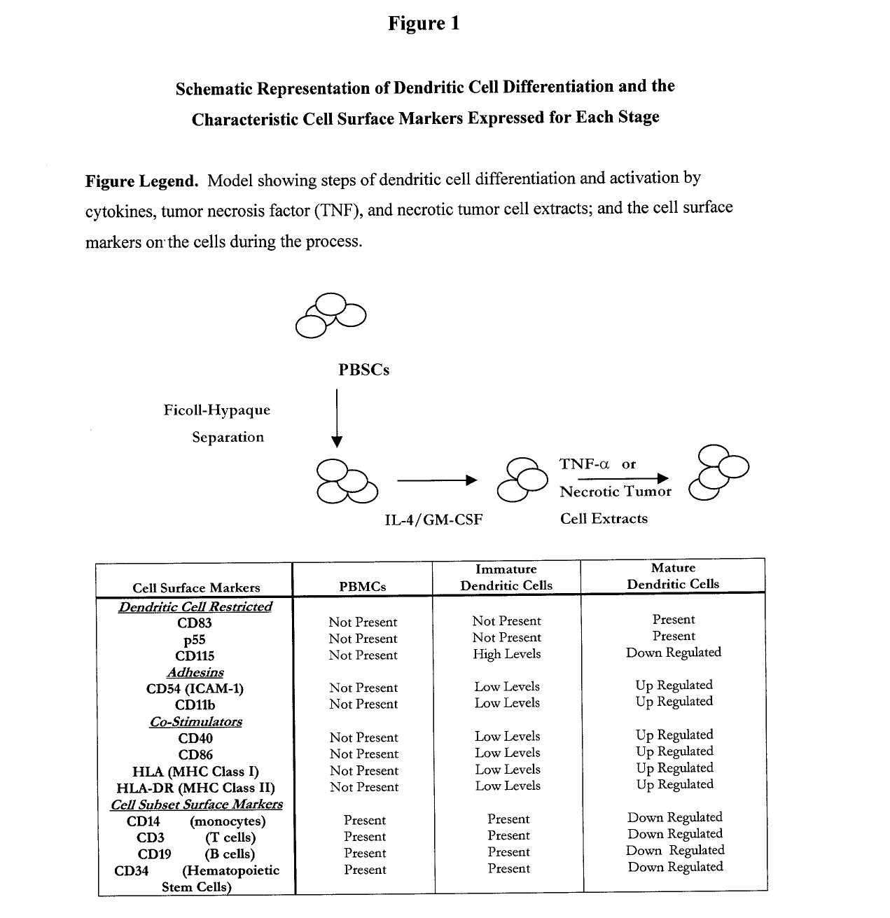

1.1. Introduction. In the following studies peripheral blood stem cells (PBSCs) were isolated (with informed consent) from a normal patient undergoing apheresis. The PBSCs were separated by Ficoll-Hypaque density gradient centrifugation and will be referenced as peripheral blood mononuclear cells (PBMCs) throughout this application. The experiments in the preliminary data and proposed in the experimental design section utilize dendritic cell activation methods and characteristics delineated in FIG. 1 (7).

1.2 Methods and Results.

1.2.1. General. PBSCs separated by Ficoll-Hypaque density gradient centrifugation from a normal donor were brought out of cryopreservation according to standard quick-thaw methods. The cells were layered over warm histopaque and centrifuged at 400×g to separate dead from viable cells. The number and viability of the cell population was then assayed by Trypan Blue Exclusion. On day zero, cells were seeded at 5×10⁶ cells into Teflon-coated bags (American Fluoroseal, Inc.) containing either standard activation media, RPMI 1640+20% fetal bovine serum or INCELL's specialty media, M3™, supplemented with 20% fetal bovine serum (i.e., M3:20™) to compare the effect each had on the rate of dendritic cell activation. The above media were either used alone or supplemented with cytokines GM-CSF (1000 U/ml) and IL-4 (500 U/ml). On day two, TNF-α (100 U/ml) was added to half of the cell population in each media type. The rate of dendritic cell activation, evaluated by the presence of dendritic cell surface markers, was monitored on aliquots collected from each bag on day zero, two, four, and seven. Cells were stained with anti-CD83 (Becton Dickinson) monoclonal antibodies in immunocytochemistry experiments.

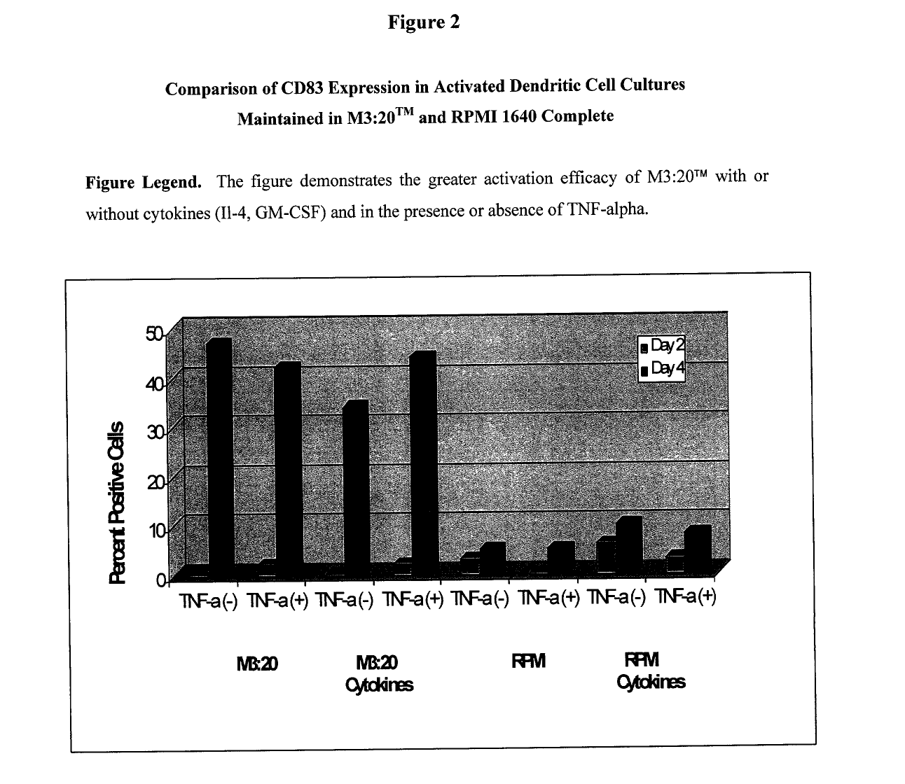

1.2.2 Marker Assays Show INCELL Medium Accelerates Dendritic Cell Activation. Marker analyses were done to compare the percentages of CD83 positive mature dendritic cells maintained in M3™ or RPMI medium (FIG. 2). A remarkably accelerated maturation of dendritic cells was observed in cells grown in INCELL's M3™ specialty medium by day four as indicated by the large population of cells which stained positive for CD83 expression. This acceleration was independent of the presence of cytokines GM-CSF and IL-4 or growth factor TNF-α. In comparison, activation of cells grown in standard activation media (RPMI 1640+20% FBS) did not occur until day seven and was dependent on cytokine supplementation (data not shown).

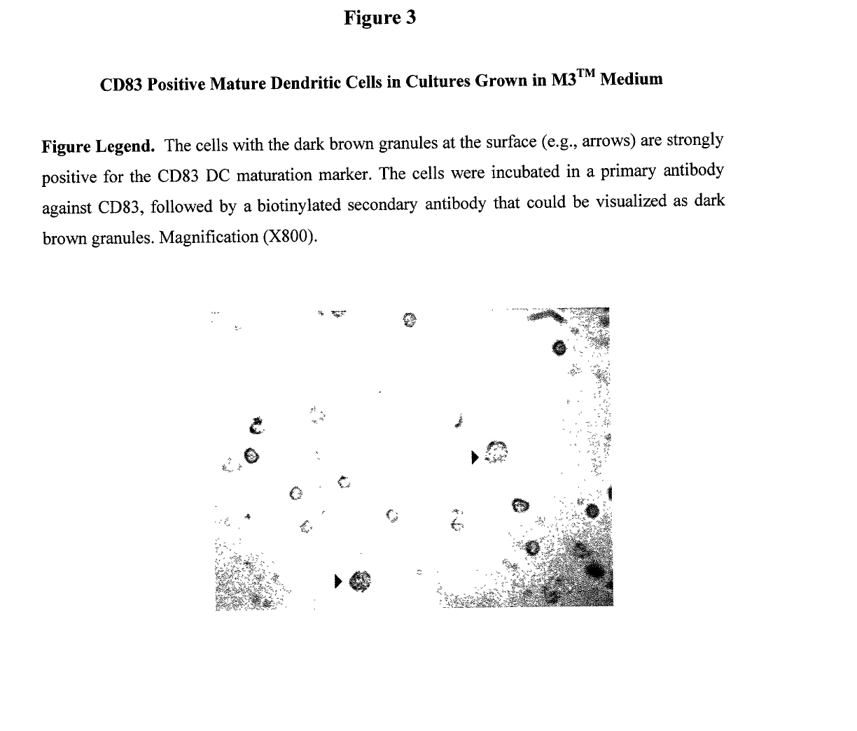

1.2.3 Morphological Changes Correlate with Marker Assays. The morphology of the cell populations in the M3™ medium exhibited large veiled cells with dendrites by day 4 (see arrows in FIG. 3). The percentages of these cells again were much higher in the M3™ medium than in RPMI 1640.

1.2.4 Long-Term Cultures Can Be Maintained in INCELL's Specialty Medium. Treatment of cell cultures with growth factors was suspended on day six. Cells were then maintained in either INCELL's M3™ specialty medium or standard activation media (RPMI 1640) alone. Within two months of activation, cells maintained in RPMI 1640 were no longer viable. These results correlate with those seen in previous studies in which cell cultures could only be maintained for a maximum of five to six weeks in RPMI 1640+10% fetal bovine serum supplemented with cytokines GM-CSF and IL-4 and growth factor TNF-α (15). In comparison, our preliminary experiments have resulted in establishment of long-term cultures of peripheral blood mononuclear cell-long term cultures (PBMC-LTC) maintained solely in INCELL's M3™ specialty medium, M3:20™.

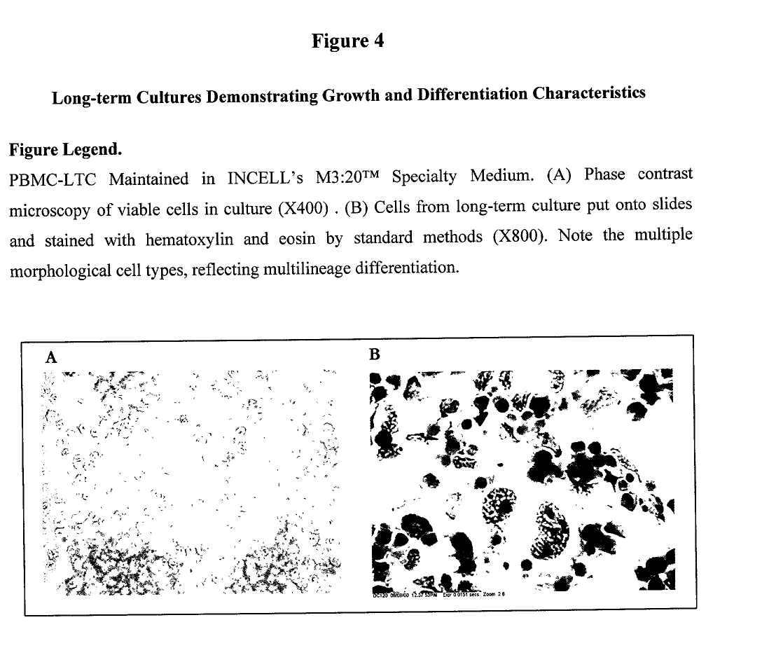

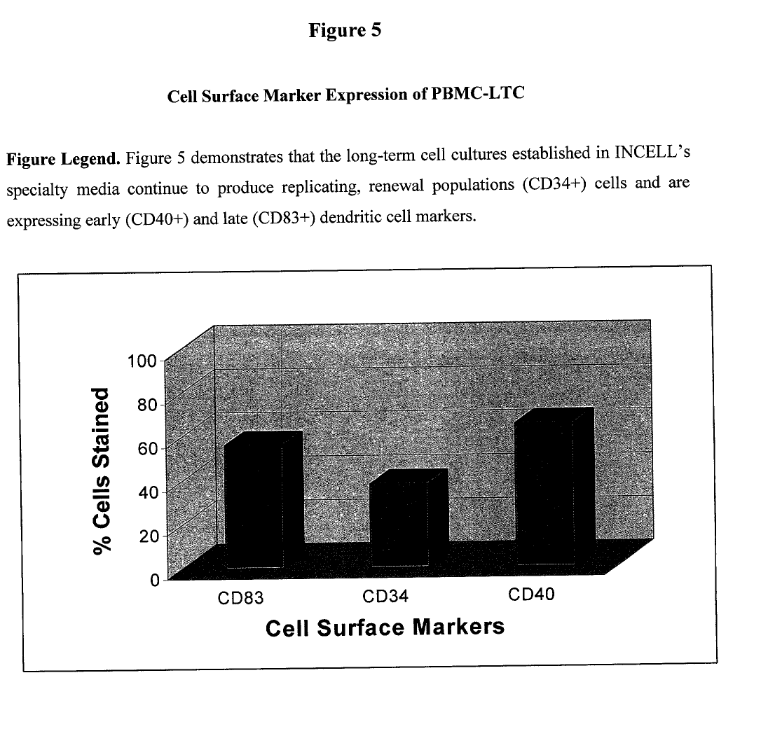

The cells shown in FIG. 4 were grown in M3:20™ and have been maintained more than a year in culture. They have cells of lymphoid, mesenchymal, and monocytic origin. When the cells are monitored for the presence of early/late dendritic cell surface markers (FIG. 5), the cultures are still positive for CD83 and CD40, indicating the presence of immature and mature dendritic cells. In addition, the cultures have reverted back to an increased CD34 expression in a large percentage of the cell population, indicating continuing cell renewal.

1.3 Conclusions: Advantages Over Current Methods.

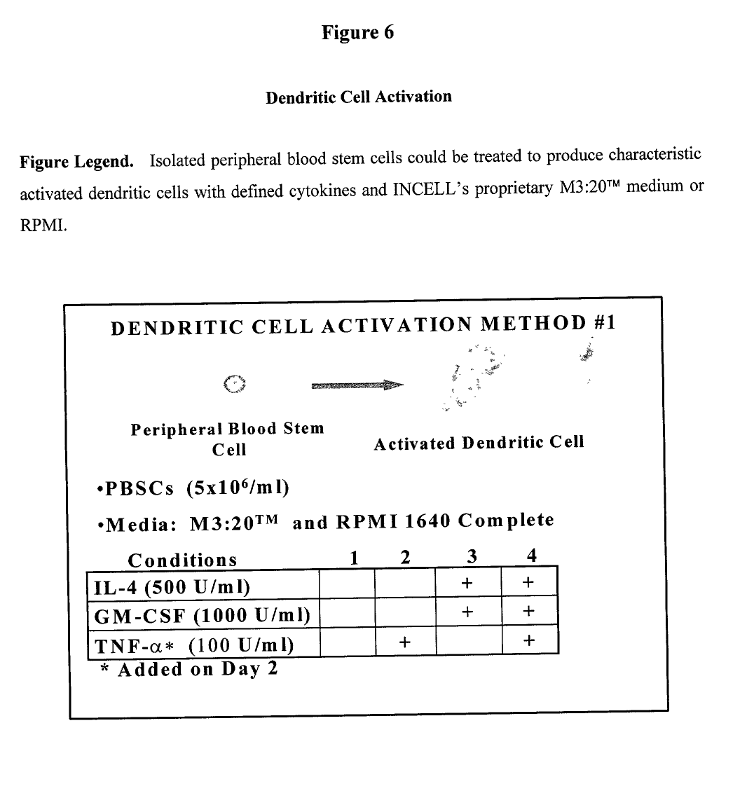

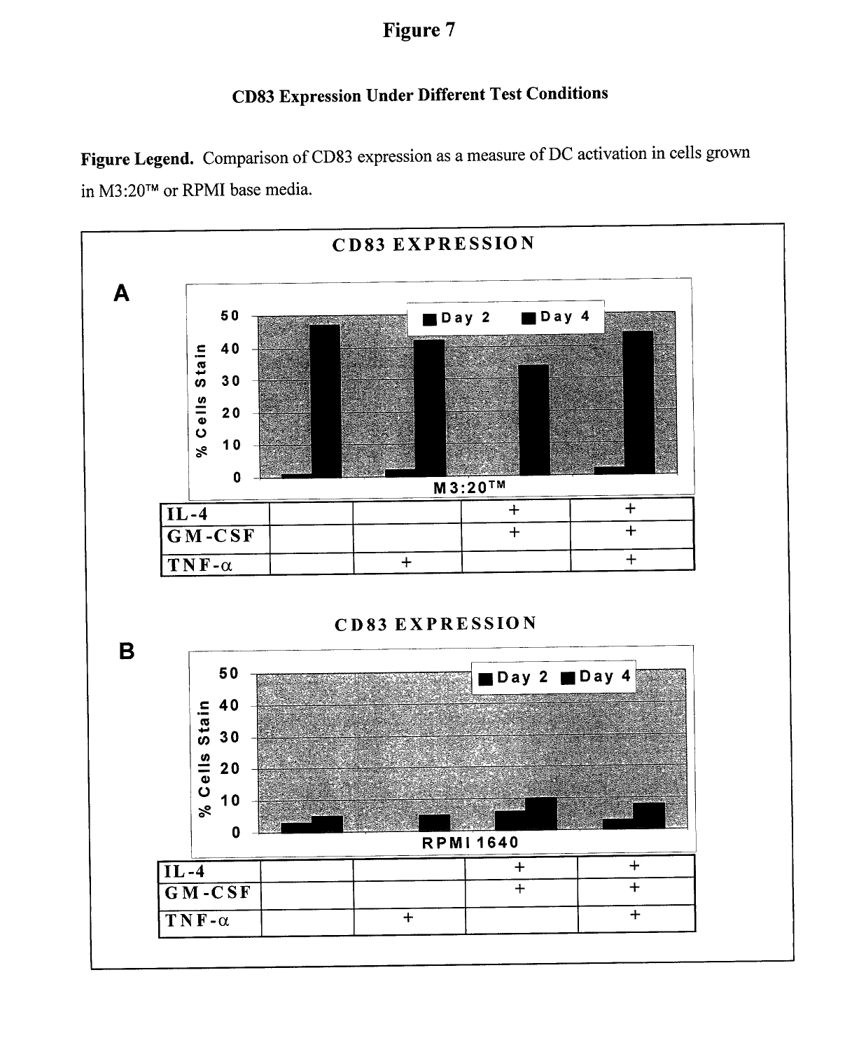

These methods briefly described in FIG. 6 are superior to currently used methods for propagation and activation of cells with potential use in transplantation or therapy. Currently, the prevalent method for activation and maturation of dendritic cells is to use RPMI 1640 media with 10-20% fetal bovine serum supplemented with various growth factors (2, 11, 15) or RPMI 1640 with 1% autologous human plasma (12, 13). This method consists of generating a population of immature dendritic cells after 6-7 days culture in RPMI complete (either 10% FBS or 1% autologous human serum) with IL-4 and GM-CSF (12). At Day 7, immature dendritic cells are stimulated to become mature dendritic cells after 2-3 days with cytokines such as TNF-α, macrophage conditioned media or necrotic tumor cell extracts (2, 12, 13, 15). Other groups report mature dendritic cell populations after 7 days incubation with RPMI complete with IL-4, GM-CSF and TNF-α (11). In contrast, our studies clearly demonstrate that PBSCs can be stimulated to become mature dendritic cells within 3-4 days (e.g., FIG. 7) when placed in INCELL's M3™ specialty medium with or without growth factors (IL-4, GM-CSF and TNF-α).

After maturation, maintenance of dendritic cells in RPMI or related activation media (with or without cytokines) has not gone beyond five weeks in most studies (15). In comparison, long-term cultures of PBMCs have been established in M3™ and have continued to grow for over a year. Preliminary evidence indicates that early/late dendritic cell surface markers, as well as population renewal markers, have continued to be expressed even in the absence of supplemental cytokines and growth factors.

M3™ is a defined medium formulation. It is expected that its use with autologous plasma will have demonstrable clinical relevance and benefit to patients requiring cell transplantation or allogeneic cell treatments for cancer or other diseases.

STUDY #2: Validation of a Dendritic Cell Activation and Function.

2.1. Introduction. In this study, DC activation and function were further validated by testing cytokines alone, and in combination with necrotic extract from breast cancer cells to activate cells obtained from a breast cancer patient.

2.2 Methods and Results.

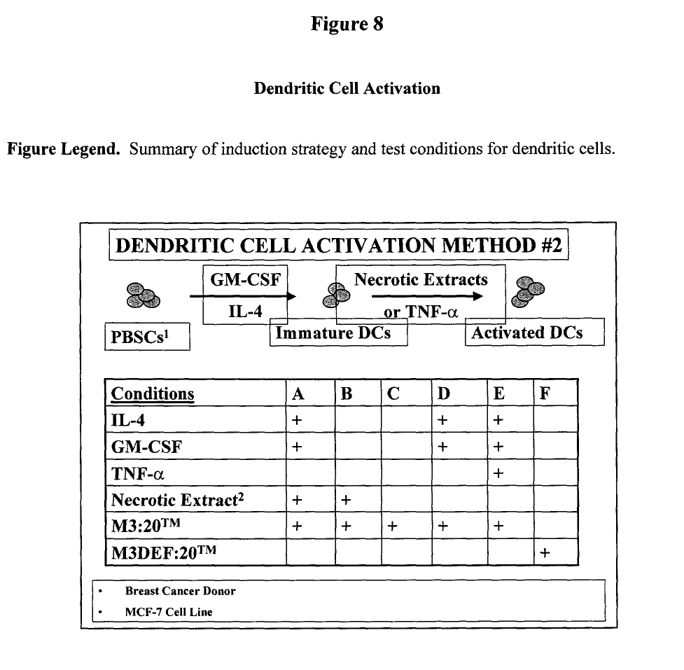

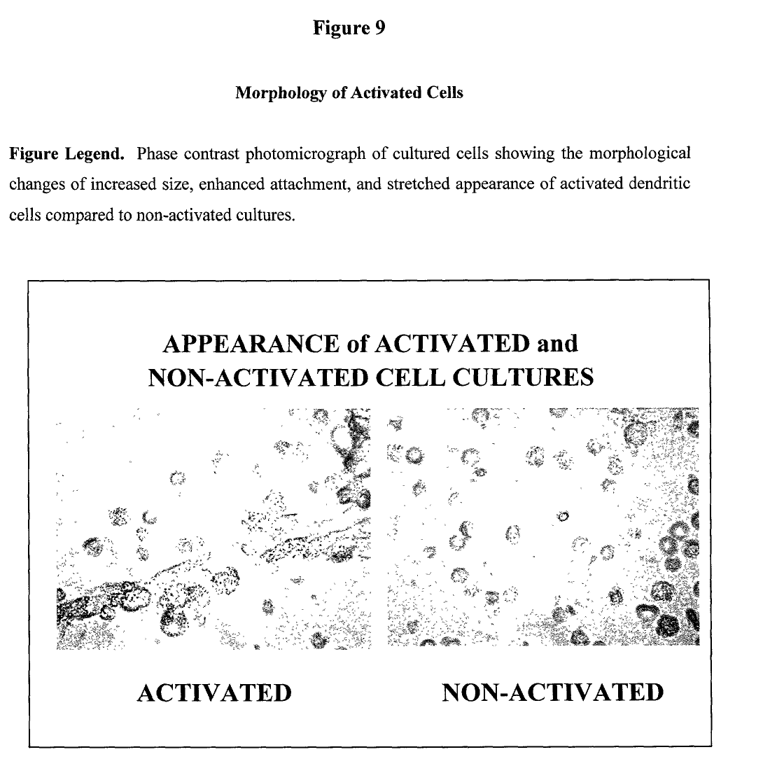

2.2.1 Cell Activation. Peripheral blood stem cell samples have been cryopreserved or obtained as fresh samples as PBSCs after G-CSF treatment of the donor. In activation experiments, the cells were seeded into medium alone or supplemented with various factors (FIG. 8). The cell population marker changes from PBSC isolation through immature DC differentiation into mature DCs was monitored by their appearance (FIG. 9) and immunocytochemistry for CD83 maturation markers (data not shown).

2.2.2 Mixed Lymphocyte Reaction. Autologous responder PBSCs (1×10⁵ cells/well) were seeded into 96-well plates. DC cell populations (72 hr post activation) were treated with Mitomycin C and added (1×10⁵ cells/well) to the responder cells (1:1 ratio). Cell proliferation was measured after 96 hrs with BrdU incorporation assay.

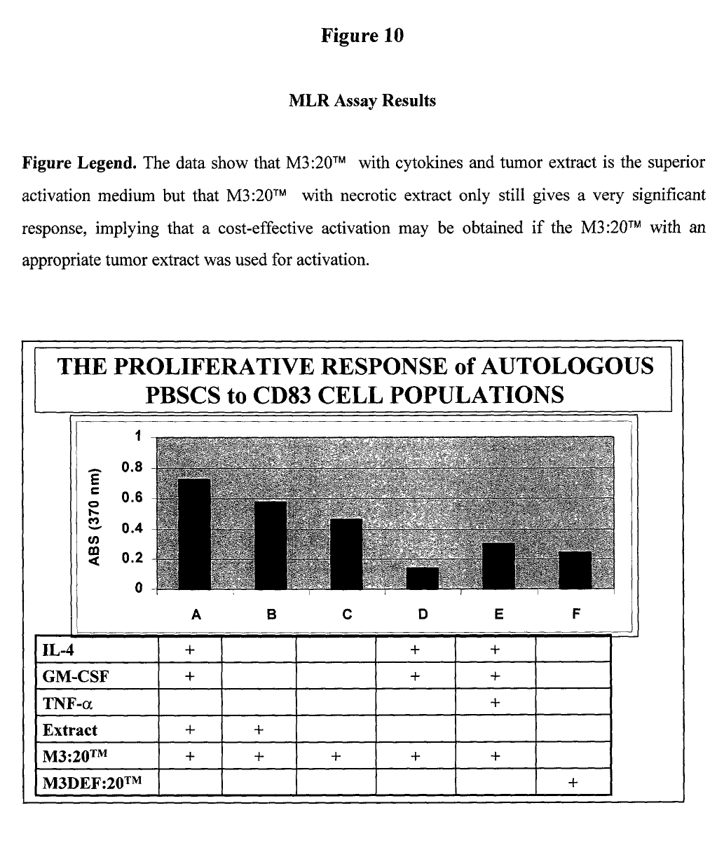

As shown in FIG. 10, M3™ supplemented medium plus tumor antigens present in the extract was the optimal method to generate a proliferative response in the MLR assays. The addition of the cytokines enhanced the response more. Thus, either method may be used to develop activated cells for use alone or as part of a kit. Marker assays for CD83 correlated with the response, verifying that DC activation followed by MLR was the mechanism of action.

2.3 Conclusions: These results show that the cells from the autologous donor were activated in an MLR against the same type of tumor (i.e., breast) and that the M3™ was superior when breast tumor cell extracts (with or without additional cytokines) were added. These exciting new methods and results, combined with Study #1, verify that methods have been developed for the propagation of human blood stem cells, activated dendritic and other immunotherapeutic cells. It further shows that the cells are bioactive when stimulated by antigens of the tumor type of the host cancer-bearing donor, validating their potential direct use for treatment regimens. To that end, process development and commercialization approaches will include the development of easy to use kits for therapeutic applications in transplantation, cancer, or other needs.

Commercialization by Development of a Prototype Dendritic Cell Activation/Maturation Kit.

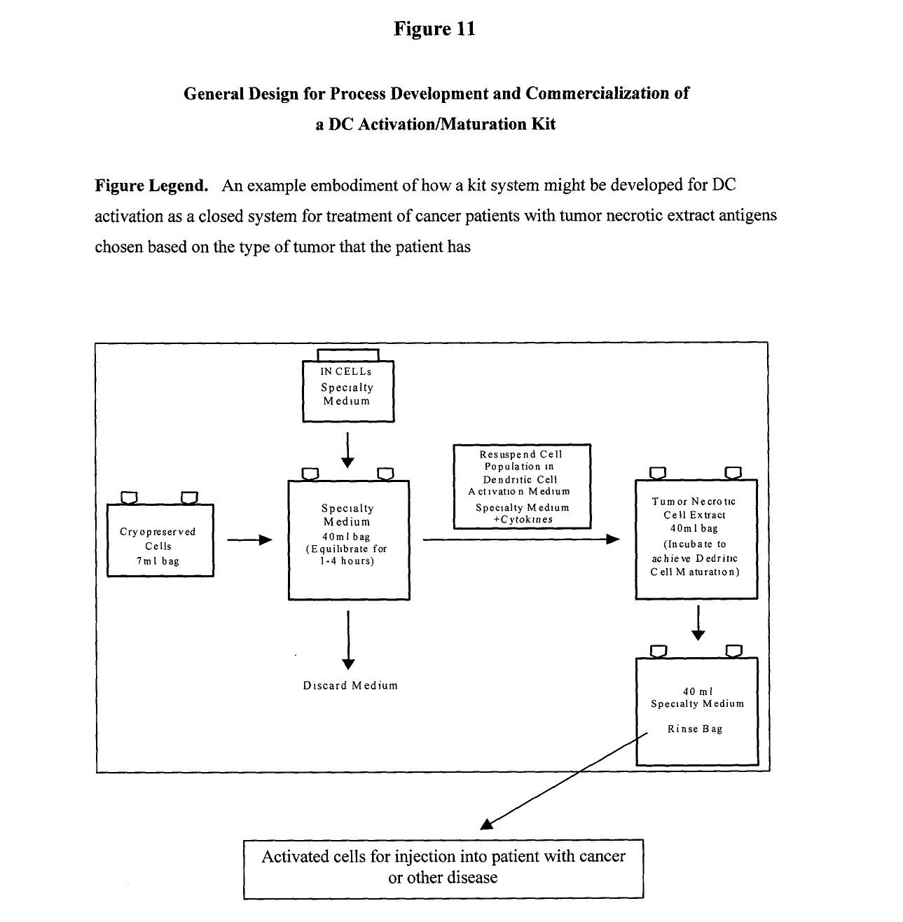

The protocols described above, as modified for use in a closed bag and Luer-lok system using INCELL's specialty media and existing approved devices, will be the basis of the kit. A simplistic view of he general components of the activation parts of the kit are shown in FIG. 11. Other modifications and connections between the systems are other possible renditions of the design. For example, the kits may be customized depending on tumor type and whether or not autologous or allogeneic cells are being used. The cells in the kit may come from peripheral blood, bone marrow or lymphoid organs as an autologous or allogeneic source.

The type and amount of antigen present would be customized for optimal activation and might come from the patient's own tumor or an allogeneic tumor or tumor cell line. The antigen concentration might range from 0.1 micrograms to 10 mg per one to 10 million cells to be activated. The antigen may be purified by column chromatography or other methods or may be a crude extract obtained from a characterized cell line or tumor extract. Alternatively, the antigen may be a defined or synthesized antigen, that might be a peptide, a carbohydrate, or a combined peptide-carbohydrate or peptide or carbohydrate attached to another molecule such as a lipid. The antigen may be produced by a vector that could include a virus, bacterium, yeast, or tissue cells of a living plant or animal organism.

1. M3™ accelerates DC proliferation and maturation.

2. M3™ promotes long-term culture of DCs and stem cells compared to standard media formulations.

3. M3™ use can shorten ex vivo manipulation time.

4. Cells can be cryopreserved and re-manipulated for bioactivity or activation after storage.

5. Tumor antigens can activate cells to become immunogenic under the test conditions.

6. These methods are applicable to breast cancer treatment.

7. These methods are applicable to treatment of other cancers, including colon, gastric, brain, liver, reproductive, prostate, melanoma, myeloma, other alimentary tract, and all others.

8. The methods developed can be applied to develop kits for clinical applications in dendritic cell transplantation and immune therapy.

9. The methods developed can be applied to develop kits for clinical applications in dendritic cell therapy.

10. The methods developed can be applied to develop kits for clinical applications in dendritic cell transplantation or therapy with other cells or treatments, including genetic and pharmaceutical therapies.

11. The methods developed can be applied to develop kits for clinical applications in dendritic cell transplantation and therapy with newly discovered cytokines, genes, or other adjuvant co-factors.

12. M3™ can be used with serum or plasma additives, including serum albumin, or autologous patient serum or plasma.

13. M3™ can be used as a modified formulation to include or delete factors that may augment or diminish immune responses of interest.

14. A defined M3™ can be used as a modified formulation.

15. The defined formulation may or may not contain serum.

16. The methods described may be applied to autologous or allogeneic cells.

17. The isolated cells can maintain the ability to grow and differentiate to dendritic cells if they are immediately cryopreserved, or if they are cultured then cryopreserved.

18. Cells obtained from peripheral blood mononuclear cells, peripheral blood stem cells, bone marrow, lymphoid organs, or other sites of dendritic cells precursors are potential sources of cells to be used for culture and activation.

19. A kit, comprised of a system of bags or other culture vessels that contain media or activation agents defined in claims 1-18, to which the cells to be activated are added, treated serially for activation, then rinsed as a prelude to their use for immune therapy.

20. Said kit of 19, which contains autologous cells or allogeneic cells, which may be universal donor cells that can be activated against multiple types of tumors or infectious agents, and which may contain a self-destruct gene or other mechanism.
